(19) United States
(12) Patent Application Publication
(10) Pub. No.: US 2022/0283160 A1
(43) Pub. Date: Sep. 8, 2022

(54) METHODS AND SYSTEMS FOR RAPID DETECTION OF SARS-COV-2 USING A RADIOLABELED ANTIBODY

(71) Applicant: MEMORIAL SLOAN KETTERING CANCER CENTER, New York, NY (US)

(72) Inventors: Giacomo Pirovano, New York, NY (US); Naga Vara Kishore Pillarsetty, New York, NY (US); Thomas Reiner, New York, NY (US)

(21) Appl. No.: 17/531,165

(22) Filed: Nov. 19, 2021

Related U.S. Application Data
(60) Provisional application No. 63/158,200, filed on Mar. 8, 2021.
(60) Provisional application No. 63/177,051, filed on Apr. 20, 2021.

(51) Int. Cl. G01N 33/569; G01N 1/10; G01N 33/60; G01N 23/00

(52) U.S. Cl. CPC G01N 33/56988; G01N 1/10; G01N 33/60; G01N 23/00; G01N 2001/1056; G01N 2333/165; G01N 2469/10

(57) ABSTRACT

The present disclosure relates to methods and kits for detecting SARS-CoV-2 virus in a patient. For example, a method is disclosed that includes contacting at least a portion of a diluted saliva sample, the diluted saliva sample including a saliva sample from a patient and a saline solution, with a radiolabeled SARS-CoV-2-targeted antibody to form a first solution that includes target bound antibody and unbound antibody; separating at least a portion of the target bound antibody from the unbound antibody in the first solution to form a separated target bound antibody solution; and detecting a radiation level in the separated target bound antibody sample indicating the presence of the target bound antibody in the separated target bound antibody sample.

### CROSS REFERENCE TO RELATED APPLICATIONS

This application claims the benefit of and priority to U.S. Provisional Patent Application No. 63/158,200, filed Mar. 8, 2021, and U.S. Provisional Patent Application No. 63/177,051, filed Apr. 20, 2021, the disclosures of each of which are hereby incorporated by reference in their entirety.

### STATEMENT REGARDING FEDERALLY SPONSORED RESEARCH

This invention was made with government support under CA008748 awarded by National Institutes of Health. The government has certain rights in the invention.

### TECHNICAL FIELD

The field of the present disclosure is methods and systems for the use of radiolabeled antibodies for detection of a SARS-CoV-2 virus.

### SUMMARY OF THE PRESENT TECHNOLOGY

In an aspect, a method for detecting SARS-CoV-2 virus in a patient is provided where the method includes:
contacting at least a portion of a diluted saliva sample (the diluted saliva sample including a saliva sample from a patient and a saline solution) with a radiolabeled SARS-CoV-2-targeted antibody to form a first solution where the first solution includes target bound antibody and unbound antibody;
separating at least a portion of the target bound antibody from the unbound antibody in the first solution to form a separated target bound antibody sample; and
detecting a radiation level in the separated target bound antibody sample indicating the presence of the target bound antibody in the separated target bound antibody sample.

In an aspect, a method for detecting SARS-CoV-2 virus in a patient is provided where the method includes:
contacting a diluted saliva sample (that includes a saliva sample from a patient diluted with a saline solution) with a radiolabeled SARS-CoV-2-targeted antibody comprising [¹²⁵I]I-CR3022 to form a first solution where the first solution includes target bound antibody and unbound antibody;
separating at least a portion of the target bound antibody from the unbound antibody in the first solution to form a separated target bound antibody sample; and
detecting with a gamma counter a radiation level in the separated target bound antibody sample indicating the presence of the target bound antibody in the separated target bound antibody sample.

In an aspect, a collection kit for collecting a saliva sample for use in a method of any aspect or embodiment disclosed herein is provided, the collection kit including:
instructions for collecting a saliva sample from a human patient and sending the saliva sample for analysis;
a sample collector for obtaining the saliva sample, the sample collector including a separation tube pretreated with saline solution; and
packaging for sending the saliva sample in the sample collector to a separate location for the analysis.

In an aspect, a process is provided that includes performing a method of any aspect or embodiment disclosed herein wherein the saliva sample of the method is the saliva sample of the sample collector of a collection kit of any aspect or embodiment disclosed herein.

### BRIEF DESCRIPTION OF THE DRAWINGS

FIGS. 1A-1D show a binding kit used to determine specificity of the radiolabeled antibody in a 96-well plate format (FIG. 1A), the experiment was performed using decreasing amounts of antibody and constant amounts of ACE2 proteins on the bottom of the plate and spike S1 proteins (FIG. 1B), decreasing amounts of radiolabeled antibody result in an increasing absorbance signal (FIG. 1C), decreasing amounts of radiolabeled antibody result in an decreasing radioactivity (FIG. 1D).

FIGS. 2A-2C show that a beads assay was used to determine sensitivity of the radiolabeled antibody (FIG. 2A), the experiment was performed using a gradient of spike proteins (FIG. 2B), and the experiment resulted in a detected sensitivity as low as 2.5 ng of spike protein (FIG. 2C).

FIG. 3 shows a step-by-step graphical explanation of one embodiment of a developed SARS-CoV-2 detection kit.

FIGS. 4A-4B show that the beads-spike complex was run through the separation kit and the target binding fraction (TBF) was measured (FIG. 4A; B=beads; S=spikes; Y=radiolabeled antibody, Freel=unlabeled iodine) and the separation kit was tested using increasing concentrations of SARS-CoV-2 and the TBF was measured (FIG. 4B).

FIG. 5A shows that the separation tubes were be primed using a 5% BSA-PBS solution (PBS containing 5% by weight bovine serum albumin) prior to use.

FIG. 5B shows a reduced non-specific binding (difference in % TBF) in saliva samples between pretreated and untreated tubes, when compared to beads samples.

### DETAILED DESCRIPTION

It is to be appreciated that certain aspects, modes, embodiments, variations and features of the present methods are described below in various levels of detail in order to provide a substantial understanding of the present technology.

In practicing the present methods, many conventional techniques in molecular biology, protein biochemistry, cell biology, microbiology and recombinant DNA are used. See, e.g., Sambrook and Russell eds. (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology.

The following terms are used throughout this disclosure as defined below.

Unless defined otherwise, all technical and scientific terms used herein generally have the same meaning as commonly understood by one of ordinary skill in the art to which this technology belongs. As used in this specification and the appended claims, the singular forms “a”, “an” and “the” include plural referents unless the content clearly dictates otherwise. For example, reference to “a cell” includes a combination of two or more cells, and the like. Generally, the nomenclature used herein and the laboratory procedures in cell culture, molecular genetics, organic chemistry, analytical chemistry, biochemistry and hybridization described below are those well-known and commonly employed in the art.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term (e.g., except where such number would be less than 0% or exceed 100% of a possible value)—for example, “about 10 wt. %” would be understood to mean “9 wt. % to 11 wt. %.” It is to be understood that when “about” precedes a term, the term is to be construed as disclosing “about” the term as well as the term without modification by “about”—for example, “about 10 wt. %” discloses “9 wt. % to 11 wt. %” as well as disclosing “10 wt. %.”

The phrase “and/or” as used in the present disclosure will be understood to mean any one of the recited members individually or a combination of any two or more thereof—for example, “A, B, and/or C” would mean “A, B, C, A and B, A and C, B and C, or the combination of A, B, and C.”

As used herein, the term “amino acid” includes naturally-occurring amino acids and synthetic amino acids, as well as amino acid analogues that function in a manner similar to the naturally-occurring amino acids. Naturally-occurring amino acids are those encoded by the genetic code, as well as those amino acids that are later modified, e.g., hydroxyproline, γ-carboxyglutamate, and O-phosphoserine. Naturally occurring amino acids include, for example, the twenty most common levorotatory (L,) amino acids normally found in mammalian proteins, i.e., alanine (Ala), arginine (Arg), asparagine (Asn), aspartic acid (Asp), cysteine (Cys), glutamine (Gin), glutamic acid (Glu), glycine (Gly), histidine (His), isoleucine (Ile), leucine (Leu), lysine (Lys), methionine (Met), phenylalanine (Phe), proline (Pro), serine (Ser), threonine (Thr), tryptophan, (Trp), tyrosine (Tyr), and valine (Val). Amino acids can be referred to herein by either their commonly known three letter symbols or by the one-letter symbols recommended by the IUPAC-IUB Biochemical Nomenclature Commission.

As used herein, the terms “polypeptide,” “peptide,” and “protein” are used interchangeably herein to mean a polymer comprising two or more amino acids joined to each other by peptide bonds or modified peptide bonds, i.e., peptide isosteres. Polypeptide refers to both short chains, commonly referred to as peptides, glycopeptides or oligomers, and to longer chains, generally referred to as proteins. Polypeptides may contain amino acids other than the 20 gene-encoded amino acids. Polypeptides include amino acid sequences modified either by natural processes, such as post-translational processing, or by chemical modification techniques that are well known in the art. In any embodiment herein, the peptides included in the compounds and complexes of the present technology may include only D-amino acids.

As used herein, a “control” is an alternative sample used in an experiment for the purpose of comparison. A control can be “positive” or “negative.”

As used herein, “radiolabel” refers to a moiety comprising a radioactive isotope of at least one element. Exemplary suitable radiolabels include but are not limited to those described herein.

As used herein, the term “sample” refers to clinical samples obtained from a subject or isolated microorganisms. In certain embodiments, a sample is obtained from a biological source (i.e., a “biological sample”), such as tissue, bodily fluid, or microorganisms collected from a subject. Sample sources include, but are not limited to, mucus, sputum, bronchial alveolar lavage (BAL), bronchial wash (BW), whole blood, bodily fluids, cerebrospinal fluid (CSF), urine, plasma, serum, or tissue.

As used herein, the terms “subject,” “individual,” or “patient” are used interchangeably and refer to an individual organism, a vertebrate, a mammal, or a human. In certain embodiments, the individual, patient or subject is a human.

The phrase “at least a portion of” will be understood by persons of ordinary skill in the art and will vary to some extent depending upon the context in which it is used. If there are uses of the term which are not clear to persons of ordinary skill in the art, given the context in which it is used, “at least a portion of” a solution means from about 0.0001 volume percent (vol. %) to about 100 vol. % of that solution at 20° C. and “at least a portion of” a composition means from about 0.0001 weight percent (wt. %) to about 100 wt. % of the total amount of that composition—for example, “at least a portion of a diluted saliva sample” would be understood to mean from about 0.0001 vol. % to about 100 vol. % of that diluted saliva sample; “separating at least a portion of the target bound antibody from the unbound antibody in the first solution” would be understood to mean separating from about 0.0001 wt. % to about 100 wt. % of the target bound antibody in the first solution from the unbound antibody in the first solution.

The terms “associated” and/or “binding” can mean a chemical or physical interaction, for example, between a compound of the present technology and a target of interest. Examples of associations or interactions include covalent bonds, ionic bonds, hydrophilic-hydrophilic interactions, hydrophobic-hydrophobic interactions and complexes. Associated can also refer generally to “binding” or “affinity” as each can be used to describe various chemical or physical interactions. Measuring binding or affinity is also routine to those skilled in the art. For example, compounds of the present technology can bind to or interact with a target of interest or precursors, portions, fragments and peptides thereof and/or their deposits.

It is to be understood that a volume ratio of different components in a composition is determined at 20° C. based on the initial volume of each individual component, not the final volume of combined components.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having “1-3 members” refers to groups having 1 member, 2 members, or 3 members; similarly, a group having “1-5 antibodies” refers to groups having 1 antibody, 2 antibodies, 3 antibodies, 4 antibodies, or 5 antibodies, and so forth.

Throughout this disclosure, various publications, patents, and published patent specifications are referenced by an identifying citation. Also within this disclosure are Arabic numerals referring to referenced citations, the full bibliographic details of which are provided preceding the claims. The disclosures of these publications, patents, and published patent specifications are hereby incorporated by reference into the present disclosure.

### Overview of the Present Technology

The severe acute respiratory syndrome coronavirus 2 (SARS-CoV-2) pandemic (1) brought increased attention to a widespread problem threatening humanity since its existence: highly contagious and lethal viral and other pathogenic infections. It became evident that in order to face this healthcare crisis, quick and efficient interventions were needed to discover and isolate the virus spread. In other words, it become important to follow the “three Ts rule”, namely, Test-Track-Treat (2, 3). To date, the widespread use of mRNA vaccines has been a great scientific success with more and more people getting vaccinated every day (4). However, the possibility of spreading new vaccine-resistant variants or new viruses remains (5). The COVID-19 pandemic has caused a quick shift in the way scientific research is conducted and shared, and the scientific community put together an incredible effort to adapt and repurpose skills and knowledge to address the challenges that we were and are still facing.

The coronavirus disease 2019 (COVID-19), i.e., the infectious disease that derives from SARS-CoV-2, has a median incubation period of about 5 days (2 to 14 days), with symptoms onset within about 12 days of infection (8 to 16 days) (25). The fast transmission of the virus is mainly due to the fact that it may occur in pre-symptomatic individuals (26) and that even asymptomatic patients contribute substantially to disease transmission (27). For these reasons, testing large fractions of the population is still a key step to understand and control the spread of the infection. To date, COVID-19 tests can be grouped as nucleic acid, serological, antigen, and ancillary tests, all of which play distinct roles in hospital, point-of-care, or large-scale population testing (28). Most antigen tests require a nasopharyngeal swab in order to probe for the nucleocapsid (N) or spike (S) proteins of SARS-CoV-2 virus via lateral flow or ELISA, and they typically have the advantage of being fairly fast (about less than an hour to complete). Ancillary tests comprise a broad category of personal devices (apps and wearable sensors) and hospital laboratory tests.

SARS-CoV-2 uses angiotensin-converting enzyme 2 (ACE2) as a receptor to enter host cells (6) through the receptor-binding domain (RBD) of the SARS-CoV spike protein (7). Nasopharyngeal and oropharyngeal swabs can be accurate gold standards for diagnosis of SARS-CoV-2 which uses Real Time reverse transcription Polymerase Chain Reaction (rRT-PCR). Unfortunately, that method of collection is slightly invasive, can cause discomfort, and requires close contact between healthcare workers and patients, which can pose a risk of transmission of the virus that necessitates use of personal protective equipment (PPE). PPE includes use of barriers (gowns, gloves, eye shields) and respiratory protection (masks, respirators) to protect mucous membranes, airways, skin, and clothing from contact with infectious agents (8). Moreover, rRT-PCR requires sterile collection tubes, time, typically in the order of one to three hours, and specialized laboratories with expensive reagents and adequate personnel (9).

Accordingly, there exists a need for more rapid and less invasive methods and systems for detecting SARS-CoV-2 and other viruses. The present technology answers this need while advantageously providing additional advantages

In an aspect of the present technology, a novel, fast, and inexpensive methods and systems for the direct detection of SARS-CoV-2 virions in saliva samples are provided. In contrast to other detection methods (23), the methods and systems of the present technology allow direct targeting of the S1 spike proteins on the surface of viable viruses using a radioactive detection output. The present method and systems may, in any embodiment disclosed herein, provide fast, simple, and reliable technology and may further be particularly applicable in low resource settings.

In an aspect, the SARS-CoV-2 detection kit includes a SARS-CoV-2-targeted antibody (CR3022) which targets Spike S1 on the viral surface. This antibody was radiolabeled with a long-lived isotope (Iodine-125) to allow the detection of bound antibody in samples with SARS-CoV-2. In any embodiment disclosed herein, a series of in vitro assays may be used to determine sensitivity and specificity and facilitate automation of the testing kit. Bound antibody may be extracted from saliva samples by including a centrifugation step and a semi-permeable membrane. In any embodiment disclosed herein, a testing kit may be further validated using SARS-CoV-2 virions.

By using the methods disclosed herein, radiosynthesis of [¹²⁵I]I-CR3022 was accomplished reliably without loss of binding. The SARS-CoV-2-sensing antibody is shown to maintain its Spike S1 affinity and to bind to as low as 2.5-5 ng of Spike protein. Bead-bound Spike S1 was used to develop a separation kit which proved to be both easy to use and inexpensive. The kit made it possible to extract bound antibody from the saliva-like sample. Validation of the separation kit using intact SARS-CoV-2 virions demonstrates that the kit can detect a viral concentration as low as 19700 PFU/mL (˜9.22% TBF) and as high as 1970000 PFU/mL (45.04% TBF).

Accordingly, also disclosed with respect to the present technology is the development and validation of a SARS-CoV-2 detection system based on the combination of a specific radiolabeled antibody and a separation membrane. The system may be comparable to other SARS-CoV-2 detection kits already approved by FDA and may be easily deployed to countries with limited resources for the diagnosis of COVID-19.

Embodiments according to the present disclosure are economical, scalable, portable, and fast, and may demonstrate reliable results with clean dispensing equipment and collection vials in a clean environment without the need for sterile equipment, vials or workspace.

Accuracy has been shown using a set of laboratory assays to illustrate the specificity of radiolabeled antibody [¹²⁵I]I-CR3022 to the Spike S1 target. Furthermore, using a beads assay, the detection at Spike S1 levels as low as 2.5-5 ng has been demonstrated. The integrated radiolabeled antibody provides for an easy and inexpensive detection kit based on the size-separation of SARS-CoV-2-bound antibody as compared to unbound antibody or other agents that could be present in the saliva sample. In any embodiment disclosed herein, the radiosynthesis reaction may be scalable and the entire kit may include Eppendorf-sized tubes that can be run in parallel to reach a high throughput where the only limitation would be the size of the tabletop centrifuge.

The average viral load of nasal swabs positive for SARS-CoV-2 is around 1.4×10⁶ copies/mL (8). The maximum load seems to be 7.11×10⁸ copies/mL (29). In the assay of the present disclosure, 19700 PFU/mL corresponds to 2.04×10⁸ copies/mL, which may be the limit of detection. Under stringent laboratory conditions qRT-PCR for COVID-19 has a limit of detection (LoD) of 500-1000 copies/mL (30). The currently approved qRT-PCR kits have LoD in range of 1000-6000 copies/mL (31). Quidel Sofia2 SARS Antigen FIA kit, an EUA antigen detection assay has an LoD of approximately 6 million in a sample collection (31). One skilled in the art would understand that while the disclosed method may have less sensitivity compared to some approved commercial technologies, one can increase the LoD of some disclosed assays by increasing the sample volume (and because we are concentrating the sample using centrifugation, volume is not a concern), reducing the non-specific binding using custom manufactured centrifugation filters and further optimizing the buffers. Improvement in these parameters can result in significant increase in improving LoD of our method and matching the sensitivity of commercially available antigen detecting kits such as Quidel Sofia2 SARS antigen FIA kit.

In any embodiment disclosed herein, a preferred isotope may be a long-lived isotope of iodine-125, traditionally used for biological assays and making the antibody suitable for long storage (¹²⁵I T_{1/2}=59.5 days). The gamma energy emission of iodine-125 is low energy (<35 keV), and therefore simple to shield with only a few centimeters of lead. Those physical characteristics make iodine-125 an ideal isotope for shipment and transportation of both the radiolabeled antibody and the filtered biospecimen.

In any embodiment disclosed herein, the hands-on time for performing testing may be extremely short (on the order of only a few minutes) and only require pipetting the saliva sample into the tube, followed by capping the tube. In any embodiment disclosed herein, the longest step may be the centrifugation step, which may require about 30 minutes.

In any embodiment disclosed herein, the test may be performed without sophisticated laboratory equipment or intensive training of the laboratory personnel. The small amount of radiation added to each tube (<0.1 μCi) makes it safe to handle, requiring just simple protective gloves.

In any embodiment disclosed herein, only a small amount of saliva sample may be needed. One skilled in the art understands that saliva, also in the form of droplets and aerosols, may be used as a valid alternative to nasopharyngeal swabs (10, 11) in many applications. Saliva has been proven to provide highly concordant results for viral detection (12), even though with a much lower sensitivity compared to rRT-PCR technology (13). Because a saliva sample may be collected and submitted by a patient themselves, PPE requirements are less stringent (14).

Looking at the kinetics of SARS-CoV-2 presence in saliva samples from patients, it has been shown that viral presence peaks during the first week from symptom onset (15). Most diagnostic tools for detection of SARS-CoV-2 infection that don't rely on rRT-PCR, are based on the detection of viral effects on the human immune system, i.e., those systems detect the presence of IgA, IgM, IgG antibodies that are produced against the virus (16), with some other examples of techniques such as Raman imaging (17) or machine learning (18).

Some embodiments of the present technology provide a simple radioactivity-based assay to measure viral particle load that can be used in a low resource, non-sterile setting and contribute toward developing rapid tests for COVID-19 or the next emerging infection. One skilled in the art will appreciate that the use of a similar strategy can be modified for a fast and reliable detection of viral loads in patient samples. Thus, one skilled in the art will further appreciate that an antibody-based kit such as the one presented here, can be modified to target a different antigen or biomarker such that the methods a systems described herein may be used for additional applications including, e.g., liquid biopsies.

In an aspect, a method for detecting SARS-CoV-2 virus in a patient is provided, the method comprising: contacting at least a portion of a diluted saliva sample, the diluted saliva sample comprising a saliva sample from a patient and a saline solution, with a radiolabeled SARS-CoV-2-targeted antibody to form a first solution comprising target bound antibody and unbound antibody; separating the target bound antibody from the unbound antibody in the first solution to form a separated target bound antibody solution; and detecting a radiation level in the separated target bound antibody sample indicating the presence of the target bound antibody in the separated target bound antibody sample.

In another aspect, a method for detecting a virus in a patient is provided, the method comprising: contacting at least a portion of a diluted saliva sample, the diluted saliva sample comprising a saliva sample from a patient and a saline solution, with a radiolabeled virus targeted antibody to form a first solution comprising target bound antibody and unbound antibody; separating at least a portion of the target bound antibody from the unbound antibody in the first solution to form a separated target bound antibody solution; and detecting a radiation level in the separated target bound antibody sample indicating the presence of the target bound antibody in the separated target bound antibody sample.

The examples herein are provided to illustrate advantages of the present technology and to further assist a person of ordinary skill in the art with preparing and/or using the present technology. The examples herein are also presented in order to more fully illustrate preferred aspects of the present technology. The examples should in no way be construed as limiting the scope of the present technology, as defined by the appended claims. The examples can include or incorporate any of the variations, aspects, or embodiments of the present technology described herein. The variations, aspects, or embodiments described above may also further each include and/or incorporate the variations of any or all other variations, aspects, or embodiments of the present technology

### EXAMPLES

As explained below, the inventors of the present technology were able to test a kit of the present technology in a biosafety level 3 laboratory. The kit was prepped and shipped the same day to the biosafety level 3 laboratory with a simple step-by-step guide on how to use it, and results obtained within the same day.

Chemicals were procured from commercial suppliers and used without further purification. 0.9% Phosphate buffered saline (PBS), Iodogen® and dichloromethane were obtained from Thermo Fisher Scientific (Waltham, Mass.). Anti-SARS-CoV-2 antibody CR3022 was obtained from Creative Biolabs (Shirley, N.Y.). Recombinant SARS-CoV-2 spike protein—S1 subunit (host cell receptor binding domain—RBD) with N-terminal histidine tag was purchased from Raybiotech (Peachtree Corners, Ga., catalog #230-01102-100). 1-micron diameter magnetic beads functionalized with Ni-NTA (Nickel-Nitrilotriacetic acid; HisPur™ Ni-NTA magnetic beads; Catalog #88831) used for bead assay were purchased from Thermo Fisher Scientific. Iodogen® (1,3,4,6-tetrachloro-3α,6α-diphenyl-glycoluril, catalog #P128600) coated glass reaction tubes were prepared by evaporating 50 μL of Iodogen® solution (50 μg, 1 mg/mL) in a borosilicate glass test tube (12×75 mm, catalog #14-961-26). PD MiniTrap G-25 columns (GE Healthcare, catalog #28918007) were preconditioned with 2 mL of PBS (Catalog #10-010-023) before using for separating radioiodinated antibody from the free radioiodine.

Radiosynthesis: Radiosynthesis was performed as described in published protocols (8). Briefly, 70 μL of PBS was added to an Iodogen (100 μg) precoated culture tube. To the resulting solution, 25 μg of CR3022 mAb (25 μL, 1.0 mg/mL) was added followed by addition of 9.25 MBq (250 μCi) of [¹²⁵I]I-NaI (in 17 μL of 0.1 N NaOH) and the mixture was allowed to react for 4 min at room temperature. For purification the crude product was loaded onto a PD MiniTrap G-25 column (GE Healthcare, catalog #28918007) which had been preconditioned with 2 mL of PBS. The radiolabeled antibody was purified using saline as eluant and fractions were collected and were used for the binding studies. The purity of the radiolabeled antibody was measured using SG-ITLC paper using 10% trifluoroacetic acid in water as eluent. The specific activity was about 8-10 mCi/mg.

In any embodiment herein of the present technology, other radioisotopes may be used so long as the radioisotopes meets requirements related to half-life, radioactivity, and availability—for example, the radioisotope may be any gamma emitting iodine isotope, including, but not limited to, iodine-131 or iodine-125.

Spike-ACE2 binding kit assay: To test antibody specificity to Spike S1, a commercially available in vitro kit was used (RayBio COVID-19 Spike-ACE2 binding assay kit II). Manufacturer instructions were followed for the reagents and sample preparation. 100 μL of each sample were added to each well in triplicate and incubated overnight at 4° C. with shaking. The solution was discarded the following day and washed 4 times in 1× wash solution. 100 μL of 1×HRP-conjugated IgG was added to each well for 1 h at room temperature with shaking. Samples were washed three times with 1× wash solution. 100 μL of TMB one-step substrate reagent was added to each well, incubated for 30 minutes at room temperature with shaking in the dark. 50 μL of stop solution were added to each well and the plate was immediately read at 450 nm in a plate reader. As a final step, the content of each well was lysed using 100 μL of 1M sodium dioxide and collected into a disposable plastic culture tube (12×75 mm). Wells were washed three times with PBS and each wash was added to the corresponding a disposable plastic culture tube (12×75 mm). Tubes were then counted on a gamma counter to detect radioactivity.

Magnetic beads assay: The assay was performed as previously described (24), but modified to by changing the concentration of SARS-CoV-2 spike Protein 51 subunit to test lower detection limits. Briefly, samples were prepared by aliquoting 20 μL of the magnetic bead slurry into a 1.5 mL Io-bind microcentrifuge tube (13-698-794; Fisher Scientific). The beads were washed by adding 380 μL of 1% PBS-BSA (PBS containing 1 by weight bovine serum albumin) and the tubes were vortexed for 5 s followed by a brief spin in a mini-centrifuge prior to placing the tubes on a magnetic rack (12321D; DynaMag™-2; ThermoFisher Scientific) for 30-45 s to isolate the magnetic beads. The SARS-CoV-2-S1 antigen was resuspended to achieve a gradient of concentrations of 2.5, 5.0, 50, 1,000 ng/mL. The washed beads were resuspended in 390 μL of PBS-BSA and the beads in all tubes except the control arm were incubated with 1 μg (10 μL) of His-tagged or biotinylated antigen for 15 min on an Eppendorf™ Thermomixer at 300 RPM at room temperature. Subsequently, the beads were washed once with 400 μL of % BSA-PBS before adding 0.1 μCi of the radiolabeled antibody ([¹²⁵I]I-CR3022) resuspended in 1% BSA-PBS. [¹²⁵I]I-CR3022 was incubated with antigen-coated beads for 30 min on a rotating mixer at room temperature. Thereafter, the beads were isolated using a magnet, and the supernatant containing unbound radioligand was aspirated with a pipette and collected in separate tubes. To remove non-specifically-bound radioligand, the beads were washed twice with 400 μL of PBS-BSA. Finally, the beads, supernatant and washes were measured for radioactivity on a gamma counter. The relative binding fractions were determined by dividing the percentage of total activity bound to magnetic beads to the total activity (beads+supernatants+wash). Separation kit: For separation, a Vivaspin 500 with 300,000 MWCO PES membrane (Sartorius #VS0152) was used to separate target-bound antibody from unbound antibody by using a tabletop centrifuge (Eppendorff) at 1,000×g for 30 minutes. Tubes were primed using 5% BSA-PBS (1,000×g for 5-10 minutes) to avoid non-specific binding. The separation kit was used as described with either the magnetic beads or in vitro virions. In any embodiment of the present technology, other methods of separation may be used including other gravity or affinity-based separation techniques.

In vitro detection of SARS-CoV-2: All work with infectious SARS-CoV-2 was performed in Institutional Biosafety Committee approved BSL3 and ABSL3 facilities at Johns Hopkins University School of Medicine using appropriate positive pressure air respirators and protective equipment. SARS-CoV-2/USA-WA1/2020 was obtained through BEI Resources, National Institute of Allergy and Infectious Diseases (NIAID) and propagated in Vero E6 TMPRSS cells (ATCC). The virus stocks were stored at −80° C. and titers were determined by tissue culture infectious dose 50 (TCID50) assay. On the day of the experiment, an aliquot of SARS-CoV-2 (1.97×106 PFU/mL) was diluted 10× in PBS. Each viral dilution (1 mL) was incubated with 0.2 μCi of [¹²⁵I]I-CR3022 for 30 min at room temperature. Sterile PBS was used as a negative control. Subsequently, the mixture was transferred to a separation unit with a 300 kDa pore size semi-permeable membrane (Vivaspin 500 as described above). The separation unit was centrifuged in a tabletop centrifuge (30 minutes at 1,000×g) and the filter was collected for detection the associated radiation using an automated gamma counter (Perkin Elmer). In the technology of the present disclosure, other methods of detection may be used, including other gamma counter techniques, sensitive photon cameras, or other scintillation devices.

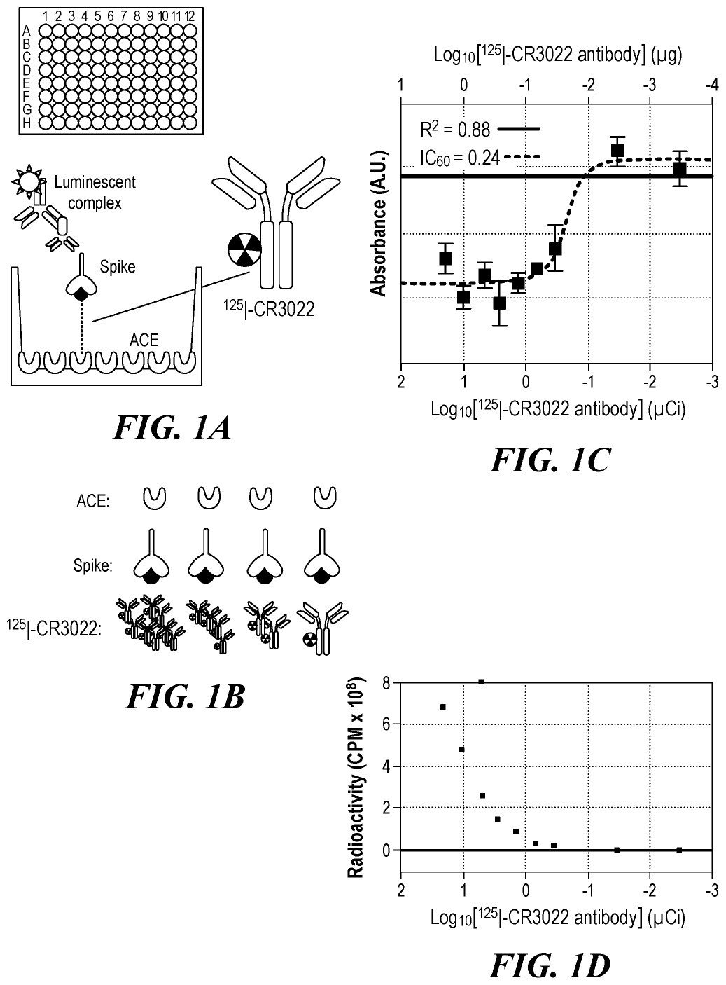

Specificity: The anti-SARS-CoV-2 51 spike antibody CR3022 was radiolabeled with a gamma-emitting iodine isotope (either iodine-131, or iodine-125) at 8-10 μCi/μg specific activity (8). In order to determine the specificity of the radiolabeled antibody to the S1 spike protein, a commercially available kit was used (RayBio COVID-19 Spike-ACE2 binding assay kit II) with ACE2 protein fixed on the bottom of the 96-well plate (FIG. 1A). The amount of ACE and Spike S1 protein was maintained constant in each well, whereas a decreasing gradient of [¹²⁵I]I-CR3022 antibody was added to inhibit the Spike-ACE interaction (FIG. 1B). Absorbance was measured at 450 nm wavelength and observed an increase determined by the [¹²⁵I]I-CR3022 decreasing gradient, confirming specificity to the Spike S1 of the radiolabeled antibody with an IC50 of 0.24 μCi≡2.4 μg (R2=0.88) as shown in FIG. 1C. After measuring absorbance, the content of each well was collected and the radioactivity measured as shown in FIG. 1D, confirming the presence of decreasing amounts of [¹²⁵I]I-CR3022.

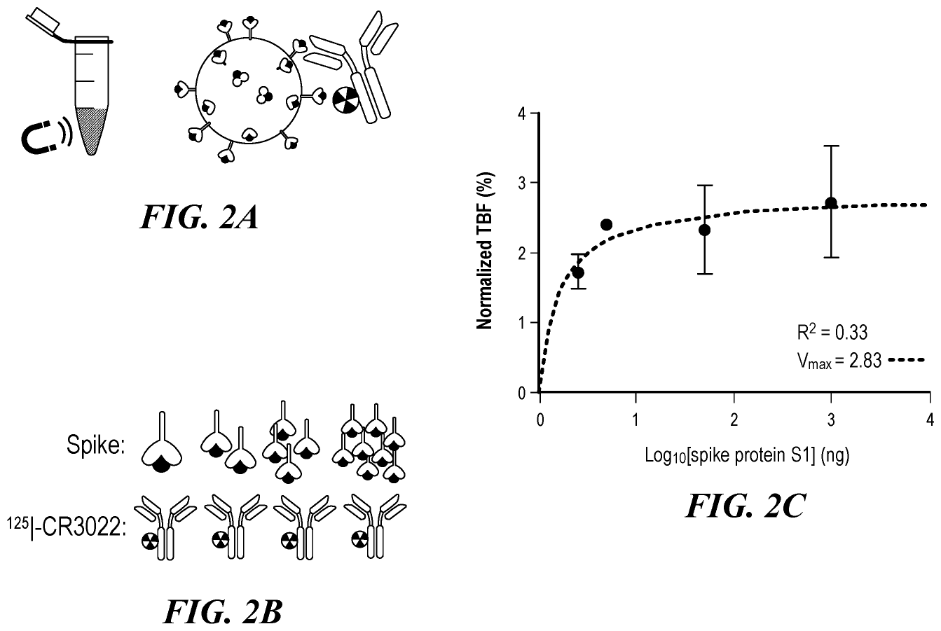

Sensitivity: In order to determine the ability to detect different amounts of Spike S1 using [¹²⁵I]I-CR3022 antibody, a sensitivity test was performed by modifying a previously published magnetic bead assay (24). To HIS-tagged Spike S1 proteins bound to magnetic beads was added the radiolabeled anti-spike antibody, as shown in FIG. 2A. An increasing gradient of Spike S1 proteins (0, 2.5, 5, 50, 1000 ng) and a constant amount of [¹²⁵I]I-CR3022 (0.1 μCi/sample, ˜0.01 μg) was used, as shown in FIG. 2B. The radiolabeled antibody was added to the beads-spike complex and then pulled-down using a magnet. The supernatant was removed. After three washes, tubes were scanned through a gamma counter to calculate the percentage target binding fraction (% TBF) as follows: % TBF=100*[CPMbeads]/[CPMbeads+CPMsupernatant+washes], where CPMbeads is the gamma counts per minute of the beads-bound activity, and CPMsupernatant+washes is the gamma counts of the supernatant and the relative washes. Counts were normalized by subtracting the CPM of beads plus no Spike S1 protein (i.e., the non-specific antibody-beads interaction). A Vmax of 2.83 was calculated by fitting the data (R2=0.33), with a curve plateau starting at about 5 ng of Spike protein, and a normalized % TBF of 1.73 at 2.5 ng as shown in FIG. 2C.

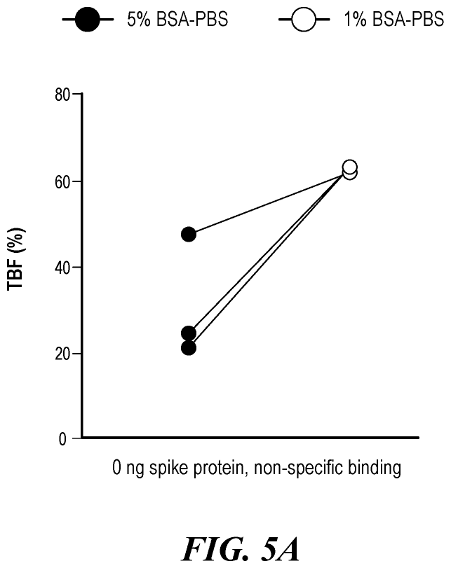

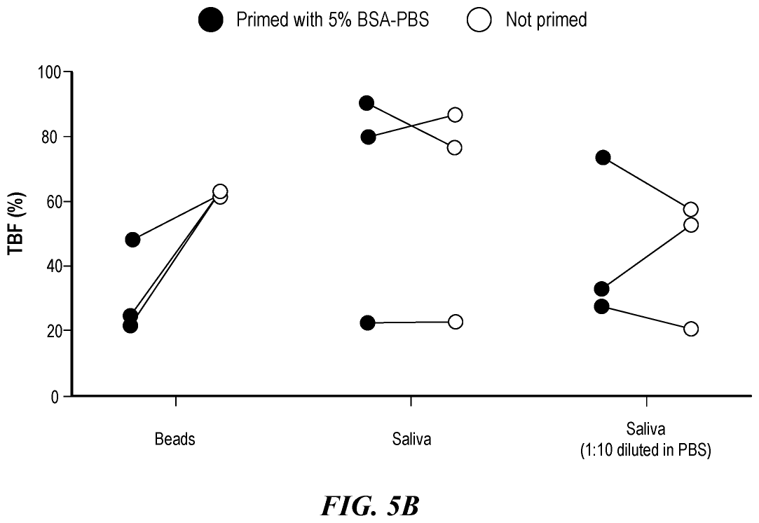

Automation: In order to be able to use the radiolabeled antibody [¹²⁵I]I-CR3022 in a more realistic scenario, a novel detection kit was developed based on the following protocol by isolating SARS-CoV-2-bound antibody based on a size exclusion step and detecting it using a gamma counter. The method used includes collecting a human biospecimen in the form of a small volume of saliva (preferably about 1 mL is sufficient, or 0.5 mL to 1.5 mL, or less than 2 ml). That sample, which might contain SARS-CoV-2 virus, is diluted in a saline solution (e.g., 1% BSA-PBS) and 500 μL of the diluted sample is added to the separation unit, a tabletop centrifuge tube with a separation membrane with a pore size of 300 kDa. In any embodiment of the present technology, less than 2000 μL, less than 1000 μL, or between about 250 μL and about 750 μL of diluted sample may be used. In any embodiment of the present technology, the tube may be primed using a 5% BSA-PBS solution prior to use as shown in FIG. 5A.

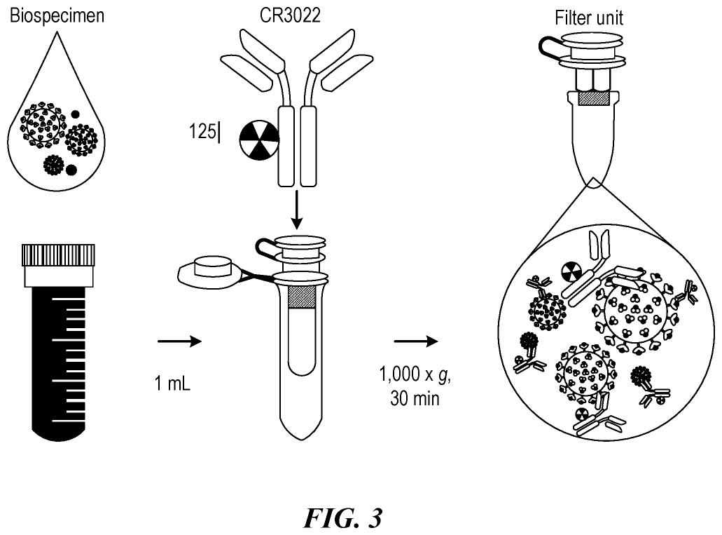

The radiolabeled antibody [¹²⁵I]I-CR3022 may then be added to the human sample directly in the separation unit. Each separation unit tube is then centrifuged in a tabletop centrifuge (30 minutes @ 1,000×g). Measuring the filter and the flow-through in a gamma counter allows detection of the amount of [¹²⁵I]I-CR3022 in each fraction and determine the % TBF as shown in FIG. 3A.

The same kit was tested by spiking saliva sample from a healthy donor to mimic human sample collection and to determine if priming tubes reduces non-specific binding in human biospecimen. A reduced non-specific binding (difference in % TBF) in saliva samples was evidenced between pretreated and untreated tubes when compared to beads samples, as shown in FIG. 5B.

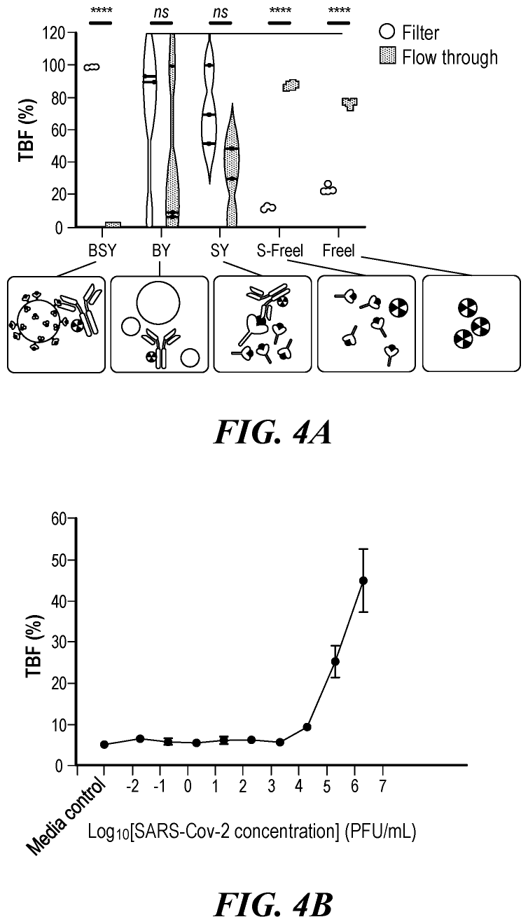

The SARS-CoV-2 detection kit was tested using Spike S1-bound magnetic beads to emulate a SARS-CoV-2 structure (despite the difference in median diameter between SARS-CoV-2˜0.2-0.05 μm and the magnetic beads˜1 μm). 500 μL of spike-carrying beads in 1% BSA-PBS were added to primed separation unit and the above protocol was followed to trap them into the filter. The % TBF was measured on a gamma counter, as described in the protocol. BSY (Spike-carrying beads with [¹²⁵I]I-CR3022 antibody) sample showed a ˜100% TBF into the filter unit, as compared to the flow though (unpaired t-test, ****p-value <0.0001). BY and SY samples (beads with [¹²⁵I]I-CR3022, and Spike S1 with [¹²⁵I]I-CR3022, respectively) did not show any significant difference between the filter-trapped and the flow-through % TBF. Free Iodine-125 with and without Spike S1 (S-Freel, and Freel, respectively) presented a significantly higher radioactive fraction flow through compared to the filter unit (unpaired t-test, ****p-value <0.0001) as shown in FIG. 4A.

Validation: In order to validate the results shown in FIG. 4A, the separation kit was tested to determine whether it could detect the presence of virulent SARS-CoV-2 in liquid samples. In vitro SARS-CoV-2 virions were diluted at different plaque-forming unit (PFU/mL) concentrations (i.e., 0.001, 0.0197, 0.197, 1.9700, 19.7000, 197.0000, 1970.0000, 19700.0000, 197000.0000, 1970000.0000 PFU/mL) in media. The kit successfully trapped into the filter unit and detected SARS-CoV-2 virions at a concentration as low as 19700 (˜9.22% TBF) and a concentration as high as 1970000 (45.04% TBF), as shown in FIG. 4B, confirming the efficacy of the kit.

As disclosed herein, the present technology provides an accurate, secure, and easy to use kit for detecting virus presence in small liquid samples containing SARSD-CoV-2 is provided. This kit may be deployed in difficult to reach areas and may significantly improve the way SARS-CoV-2 infection is tested.

### PUBLICATIONS CITED IN THE PRESENT DISCLOSURE

(1) Cucinotta D, Vanelli M. WHO Declares COVID-19 a Pandemic. Acta Biomed. 2020; 91(1):157-60. Epub 2020 Mar. 20. doi: 10.23750/abm.v91i1.9397. PubMed PMID: 32191675; PMCID: PMC7569573.
(2) Willem L, Abrams S, Libin P J K, Coletti P, Kuylen E, Petrof O, Møgelmose S, Wambua J, Herzog S A, Faes C, Beutels P, Hens N. The impact of contact tracing and household bubbles on deconfinement strategies for COVID-19. Nature Communications. 2021; 12(1):1524. doi: 10.1038/s41467-021-21747-7.
(3) Munzert S, Selb P, Gohdes A, Stoetzer L F, Lowe W. Tracking and promoting the usage of a COVID-19 contact tracing app. Nature Human Behaviour. 2021; 5(2):247-55. doi: 10.1038/s41562-020-01044-x.
(4) Rolla G, Brussino L, Badiu I. Maintaining Safety with SARS-CoV-2 Vaccines. N Engl J Med. 2021; 384(10):e37. Epub 2021 Feb. 11. doi: 10.1056/NEJMc2100766. PubMed PMID: 33567189.
(5) Carvalho T, Krammer F, Iwasaki A. The first 12 months of COVID-19: a timeline of immunological insights. Nat Rev Immunol. 2021. Epub 2021 Mar. 17. doi: 10.1038/s41577-021-00522-1. PubMed PMID: 33723416.
(6) Zhou P, Yang X L, Wang X G, Hu B, Zhang L, Zhang W, Si H R, Zhu Y, Li B, Huang C L, Chen H D, Chen J, Luo Y, Guo H, Jiang R D, Liu M Q, Chen Y, Shen X R, Wang X, Zheng X S, Zhao K, Chen Q J, Deng F, Liu L L, Yan B, Zhan F X, Wang Y Y, Xiao G F, Shi Z L. A pneumonia outbreak associated with a new coronavirus of probable bat origin. Nature. 2020; 579(7798):270-3. Epub 2020 Feb. 6. doi: 10.1038/s41586-020-2012-7. PubMed PMID: 32015507; PMCID: PMC7095418.
(7) Letko M, Marzi A, Munster V. Functional assessment of cell entry and receptor usage for SARS-CoV-2 and other lineage B betacoronaviruses. Nat Microbiol. 2020; 5(4):562-9. Epub 2020 Feb. 26. doi: 10.1038/s41564-020-0688-y. PubMed PMID: 32094589; PMCID: PMC7095430.
(8) Pillarsetty N, Carter L M, Lewis J S, Reiner T. Oncology-Inspired Treatment Options for COVID-19. Journal of nuclear medicine: official publication, Society of Nuclear Medicine. 2020; 61(12):1720-3. Epub 2020 Jul. 19. doi: 10.2967/jnumed.120.249748. PubMed PMID: 32680924.
(9) Nagura-Ikeda M, Imai K, Tabata S, Miyoshi K, Murahara N, Mizuno T, Horiuchi M, Kato K, Imoto Y, Iwata M, Mimura S, Ito T, Tamura K, Kato Y. Clinical Evaluation of Self-Collected Saliva by Quantitative Reverse Transcription-PCR (RT-qPCR), Direct RT-qPCR, Reverse Transcription-Loop-Mediated Isothermal Amplification, and a Rapid Antigen Test To Diagnose COVID-19. J Clin Microbiol. 2020; 58(9). Epub 2020 Jul. 9. doi: 10.1128/JCM.01438-20. PubMed PMID: 32636214; PMCID: PMC7448663.
(10) Azzi L, Carcano G, Gianfagna F, Grossi P, Dalla Gasperina D, Genoni A, Fasano M, Sessa F, Tettamanti L, Carinci F, Maurino V, Rossi A, Tagliabue A, Baj A. Saliva is a reliable tool to detect SARS-CoV-2. J Infection. 2020; 81(1):E45-E50. doi: 10.1016/j.jinf.2020.04.005. PubMed PMID: WOS:000540819000009.
(11) Xu R S, Cui B M, Duan X B, Zhang P, Zhou X D, Yuan Q. Saliva: potential diagnostic value and transmission of 2019-nCoV. Int J Oral Sci. 2020; 12(1). doi: 10.1038/s41368-020-0080-z. PubMed PMID: WOS:000526338700001.
(12) To K K W, Yip C C Y, Lai C Y W, Wong C K H, Ho D T Y, Pang P K P, Ng A C K, Leung K H, Poon R W S, Chan K H, Cheng V C C, Hung I F N, Yuen K Y. Saliva as a diagnostic specimen for testing respiratory virus by a point-of-care molecular assay: a diagnostic validity study. Clin Microbiol Infec. 2019; 25(3):372-8. doi: 10.1016/j.cmi.2018.06.009. PubMed PMID: WOS:000459953000017.
(13) Dinnes J, Deeks J J, Adriano A, Berhane S, Davenport C, Dittrich S, Emperador D, Takwoingi Y, Cunningham J, Beese S, Dretzke J, di Ruffano L F, Harris I M, Price M J, Taylor-Phillips S, Hooft L, Leeflang M M G, Spijker R, Van den Bruel A, Ac CC-DT. Rapid, point-of-care antigen and molecular-based tests for diagnosis of SARS-CoV-2 infection (Review). Cochrane Db Syst Rev. 2020(8). doi: 10.1002/14651858.Cd013705. PubMed PMID: WOS:000568684200005.
(14) Kapoor P, Chowdhry A, Kharbanda O P, Bablani Popli D, Gautam K, Saini V. Exploring salivary diagnostics in COVID-19: a scoping review and research suggestions. BDJ Open. 2021; 7(1):8. Epub 2021/01/28. doi: 10.1038/s41405-021-00064-7. PubMed PMID: 33500385; PMCID: PMC7836040.
(15) To K K W, Tsang O T Y, Leung W S, Tam A R, Wu T C, Lung D C, Yip C C Y, Cai J P, Chan J M C, Chik T S H, Lau D P L, Choi C Y C, Chen L L, Chan W M, Chan K H, Ip J D, Ng ACK, Poon R W S, Luo C T, Cheng V C C, Chan J F W, Hung I F N, Chen Z W, Chen H L, Yuen K Y. Temporal profiles of viral load in posterior oropharyngeal saliva samples and serum antibody responses during infection by SARS-CoV-2: an observational cohort study. Lancet Infect Dis. 2020; 20(5):565-74. doi: 10.1016/S1473-3099(20)30196-1. PubMed PMID: WOS:000530702900049.
(16) Younes N, Al-Sadeq D W, Al-Jighefee H, Younes S, Al-Jamal O, Daas H I, Yassine H M, Nasrallah G K. Challenges in Laboratory Diagnosis of the Novel Coronavirus SARS-CoV-2. Viruses-Basel. 2020; 12(6). doi: 10.3390/v12060582. PubMed PMID: WOS:000551098300001.
(17) Carlomagno C, Bertazioli D, Gualerzi A, Picciolini S, Banfi P I, Lax A, Messina E, Navarro J, Bianchi L, Caronni A, Marenco F, Monteleone S, Arienti C, Bedoni M. COVID-19 salivary Raman fingerprint: innovative approach for the detection of current and past SARS-CoV-2 infections. Scientific Reports. 2021; 11(1):4943. doi: 10.1038/s41598-021-84565-3.
(18) Roberts M, Driggs D, Thorpe M, Gilbey J, Yeung M, Ursprung S, Aviles-Rivero A I, Etmann C, McCague C, Beer L, Weir-McCall J R, Teng Z, Gkrania-Klotsas E, Ruggiero A, Korhonen A, Jefferson E, Ako E, Langs G, Gozaliasl G, Yang G, Prosch H, Preller J, Stanczuk J, Tang J, Hofmanninger J, Babar J, Sánchez L E, Thillai M, Gonzalez P M, Teare P, Zhu X, Patel M, Cafolla C, Azadbakht H, Jacob J, Lowe J, Zhang K, Bradley K, Wassin M, Holzer M, Ji K, Ortet M D, Ai T, Walton N, Lio P, Stranks S, Shadbahr T, Lin W, Zha Y, Niu Z, Rudd JHF, Sala E, Schönlieb C-B, Aix C. Common pitfalls and recommendations for using machine learning to detect and prognosticate for COVID-19 using chest radiographs and CT scans. Nature Machine Intelligence. 2021; 3(3):199-217. doi: 10.1038/s42256-021-00307-0.
(19) Wilson T C, Jannetti S A, Guru N, Pillarsetty N, Reiner T, Pirovano G. Improved radiosynthesis of (123)I-MAPi, an auger theranostic agent. Int J Radiat Biol. 2020:1-7. Epub 2020 Jun. 20. doi: 10.1080/09553002.2020.1781283. PubMed PMID: 32552309; PMCID: PMC7775866.
(20) Pirovano G, Jannetti S A, Carter L M, Sadique A, Kossatz S, Guru N, Demetrio De Souza Franca P, Maeda M, Zeglis B M, Lewis J S, Humm J L, Reiner T. Targeted Brain Tumor Radiotherapy Using an Auger Emitter. Clin Cancer Res. 2020; 26(12):2871-81. Epub 2020 Feb. 19. doi: 10.1158/1078-0432.CCR-19-2440. PubMed PMID: 32066626; PMCID: PMC7299758.
(21) Jannetti S A, Carlucci G, Carney B, Kossatz S, Shenker L, Carter L M, Salinas B, Brand C, Sadique A, Donabedian P L, Cunanan K M, Gonen M, Ponomarev V, Zeglis B M, Souweidane M M, Lewis J S, Weber W A, Humm J L, Reiner T. PARP-1-Targeted Radiotherapy in Mouse Models of Glioblastoma. J Nucl Med. 2018; 59(8):1225-33. Epub 2018 Mar. 25. doi: 10.2967/jnumed.117.205054. PubMed PMID: 29572254; PMCID: PMC6071508.
(22) Gangangari K K, Varadi A, Majumdar S, Larson S M, Pasternak G W, Pillarsetty N K. Imaging Sigma-1 Receptor (S1R) Expression Using Iodine-124-Labeled 1-(4-Iodophenyl)-3-(2-adamantyl)guanidine ([(124)I]IPAG). Mol Imaging Biol. 2020; 22(2):358-66. Epub 2019 Jun. 6. doi: 10.1007/s11307-019-01369-8. PubMed PMID: 31165385; PMCID: PMC6893110.
(23) Sethuraman N, Jeremiah S S, Ryo A. Interpreting Diagnostic Tests for SARS-CoV-2. JAMA. 2020; 323(22):2249-51. Epub 2020 May 7. doi: 10.1001/jama.2020.8259. PubMed PMID: 32374370.
(24) Sharma S K, Lyashchenko S K, Park H A, Pillarsetty N, Roux Y, Wu J, Poty S, Tully K M, Poirier J T, Lewis J S. A rapid bead-based radioligand binding assay for the determination of target-binding fraction and quality control of radiopharmaceuticals. Nucl Med Biol. 2019; 71:32-8. Epub 2019 May 28. doi: 10.1016/j.nucmedbio.2019.04.005. PubMed PMID: 31128476; PMCID: PMC6599726.
(25) Lauer S A, Grantz K H, Bi Q, Jones F K, Zheng Q, Meredith H R, Azman A S, Reich N G, Lessler J. The Incubation Period of Coronavirus Disease 2019 (COVID-19) From Publicly Reported Confirmed Cases: Estimation and Application. Ann Intern Med. 2020; 172(9):577-82. Epub 2020 Mar. 10. doi: 10.7326/M20-0504. PubMed PMID: 32150748; PMCID: PMC7081172.
(26) He X, Lau EHY, Wu P, Deng X, Wang J, Hao X, Lau Y C, Wong J Y, Guan Y, Tan X, Mo X, Chen Y, Liao B, Chen W, Hu F, Zhang Q, Zhong M, Wu Y, Zhao L, Zhang F, Cowling B J, Li F, Leung G M. Temporal dynamics in viral shedding and transmissibility of COVID-19. Nat Med. 2020; 26(5):672-5. Epub 2020 Apr. 17. doi: 10.1038/s41591-020-0869-5. PubMed PMID: 32296168.
(27) Li R, Pei S, Chen B, Song Y, Zhang T, Yang W, Shaman J. Substantial undocumented infection facilitates the rapid dissemination of novel coronavirus (SARS-CoV-2). Science. 2020; 368(6490):489-93. Epub 2020 Mar. 18. doi: 10.1126/science.abb3221. PubMed PMID: 32179701; PMCID: PMC7164387.
(28) Weissleder R, Lee H, Ko J, Pittet M J. COVID-19 diagnostics in context. Sci Transl Med. 2020; 12(546). Epub 2020 Jun. 5. doi: 10.1126/scitranslmed.abc1931. PubMed PMID: 32493791.
(29) Wolfel R, Corman V M, Guggemos W, Seilmaier M, Zange S, Muller M A, Niemeyer D, Jones T C, Vollmar P, Rothe C, Hoelscher M, Bleicker T, Brunink S, Schneider J, Ehmann R, Zwirglmaier K, Drosten C, Wendtner C. Virological assessment of hospitalized patients with COVID-2019. Nature. 2020; 581(7809):465-9. Epub 2020 Apr. 3. doi: 10.1038/541586-020-2196-x. PubMed PMID: 32235945.
(30) Zhang Y, Dai C, Wang H, Gao Y, Li T, Fang Y, Shen Z, Chen L, Chen Z, Ma X, Li M. Analysis and validation of a highly sensitive one-step nested quantitative real-time polymerase chain reaction assay for specific detection of severe acute respiratory syndrome coronavirus 2. Virol J. 2020; 17(1):197. Epub 2020 Dec. 30. doi: 10.1186/s12985-020-01467-y. PubMed PMID: 33371898; PMCID: PMC7768088.
(31) Arnaout R, Lee R A, Lee G R, Callahan C, Yen C F, Smith K P, Arora R, Kirby J E. SARS-CoV2 Testing: The Limit of Detection Matters. bioRxiv. 2020. Epub 2020 Jun. 25. doi: 10.1101/2020.06.02.131144. PubMed PMID: 32577640; PMCID: PMC7302192.
(32) Wu Q, Suo C, Brown T, Wang T, Teichmann S A, Bassett A R. INSIGHT: A population-scale COVID-19 testing strategy combining point-of-care diagnosis with centralized high-throughput sequencing. Sci Adv. 2021; 7(7). Epub 2021 Feb. 14. doi: 10.1126/sciadv.abe5054. PubMed PMID: 33579697; PMCID: PMC7880595.

While certain embodiments have been illustrated and described, a person with ordinary skill in the art, after reading the foregoing specification, can effect changes, substitutions of equivalents and other types of alterations to the compounds and complexes of the present technology as set forth herein. Each aspect and embodiment described above can also have included or incorporated therewith such variations or aspects as disclosed in regard to any or all of the other aspects and embodiments.

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds, compositions, or biological systems, which can, of course, vary. It is also to be understood that the terminology used herein is for the purpose of describing particular embodiments only, and is not intended to be limiting.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

In addition, where features or aspects of the disclosure are described in terms of Markush groups, those skilled in the art will recognize that the disclosure is also thereby described in terms of any individual member or subgroup of members of the Markush group. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents (for example, journals, articles and/or textbooks) referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

The present technology may include, but is not limited to, the features and combinations of features recited in the following lettered paragraphs, it being understood that the following paragraphs should not be interpreted as limiting the scope of the claims as appended hereto or mandating that all such features must necessarily be included in such claims:
A. A method for detecting SARS-CoV-2 virus in a patient, the method comprising:
   contacting at least a portion of a diluted saliva sample, the diluted saliva sample comprising a saliva sample from a patient and a saline solution, with a radiolabeled SARS-CoV-2-targeted antibody to form a first solution comprising target bound antibody and unbound antibody;
   separating at least a portion of the target bound antibody from the unbound antibody in the first solution to form a separated target bound antibody sample; and
   detecting a radiation level in the separated target bound antibody sample indicating the presence of the target bound antibody in the separated target bound antibody sample.
B. The method of Paragraph A, wherein the radiolabeled SARS-CoV-2-targeted antibody comprises a radioisotope of iodine, optionally where the radioisotope of iodine comprises iodine-123, iodine-124, iodine-125, and/or iodine-131.
C. The method of Paragraph A or Paragraph B, wherein the radiolabeled SARS-CoV-2-targeted antibody targets Spike S1 of SARS-CoV-2.
D. The method of any one of Paragraphs A-C, wherein the SARS-CoV-2-targeted antibody comprises CR3022.
E. The method of any one of Paragraphs A-D, wherein the radiolabeled SARS-CoV-2-targeted antibody comprises [¹²⁵I]I-CR3022.
F. The method of any one of Paragraphs A-E, wherein the radiolabeled SARS-CoV-2-targeted antibody comprises iodine-125.
G. The method of any one of Paragraphs A-F, wherein the diluted saliva sample comprises a volume ratio of salvia to the saline solution of between 1:7 to 1:11.
H. The method of any one of Paragraphs A-G, wherein the diluted saliva sample comprises a volume ratio of salvia to the saline solution of about 1:9.
I. The method of any one of Paragraphs A-H, wherein the saline solution comprises 1% BSA-PBS.
J. The method of any one of Paragraphs A-I, wherein the portion of a diluted saliva sample comprises about 500 μl diluted saliva sample.
K. The method of any one of Paragraphs A-J, wherein separating at least a portion of the target bound antibody from the unbound antibody in the first solution comprises gravity separation.
L. The method of any one of Paragraphs A-K, wherein separating at least a portion of the target bound antibody from the unbound antibody in the first solution comprises centrifugal separation.
M. The method of any one of Paragraphs A-L, comprising detecting a radiation level in the separated target bound antibody solution with a gamma counter.
N. A method for detecting SARS-CoV-2 virus in a patient, the method comprising:
   contacting a diluted saliva sample comprising a saliva sample from a patient diluted with a saline solution, with a radiolabeled SARS-CoV-2-targeted antibody comprising [¹²⁵I]I-CR3022 to form a first solution comprising target bound antibody and unbound antibody;
   separating at least a portion of the target bound antibody from the unbound antibody in the first solution to form a separated target bound antibody sample; and
   detecting with a gamma counter a radiation level in the separated target bound antibody sample indicating the presence of the target bound antibody in the separated target bound antibody sample.
O. The method of Paragraph N, wherein the contacting the diluted saliva sample with the radiolabeled SARS-CoV-2-targeted antibody comprises contacting the diluted saliva sample with the radiolabeled SARS-CoV-2-targeted antibody in a separation tube, and the separating at least a portion of the target bound antibody from the unbound antibody in the first solution comprises separating at least a portion of the target bound antibody from the unbound antibody in the first solution in the separation tube using a centrifuge.
P. The method of Paragraph 0, wherein the separation tube comprises a prepacked, single-use column.
Q. The method of Paragraph 0 or Paragraph P, wherein the separation tube is pretreated with a solution comprising 5% BSA-PBS.
R. A collection kit for collecting a saliva sample for use in the method of any one of Paragraphs A-Q, the collection kit comprising:
   instructions for collecting a saliva sample from a human patient and sending the saliva sample for analysis;
   a sample collector for obtaining the saliva sample, the sample collector comprising a separation tube pretreated with saline solution; and
   packaging for sending the saliva sample in the sample collector to a separate location for the analysis.
S. The collection kit of Paragraph R, wherein the instructions for collecting the saliva sample from a human patient and sending the saliva sample for the analysis comprise instructions for contacting the saliva sample with the saline solution of the separation tube.
T. The collection kit of Paragraph R or Paragraph S, wherein the collection kit further comprises instructions for the analysis, the instructions for the analysis comprising instructions for performing the method of any one of Paragraphs A-Q.
U. A process comprising performing the method of any one of Paragraphs A-Q wherein the saliva sample of the method is the saliva sample in the sample collector of any one of Paragraphs R-U.

Other embodiments are set forth in the following claims, along with the full scope of equivalents to which such claims are entitled.

What is claimed is:

1. A method for detecting SARS-CoV-2 virus in a patient, the method comprising:
contacting at least a portion of a diluted saliva sample, the diluted saliva sample comprising a saliva sample from a patient and a saline solution, with a radiolabeled SARS-CoV-2-targeted antibody to form a first solution comprising target bound antibody and unbound antibody;
separating at least a portion of the target bound antibody from the unbound antibody in the first solution to form a separated target bound antibody sample; and
detecting a radiation level in the separated target bound antibody sample indicating the presence of the target bound antibody in the separated target bound antibody sample.

2. The method of claim 1, wherein the radiolabeled SARS-CoV-2-targeted antibody comprises a radioisotope of iodine.

3. The method of claim 1, wherein the radiolabeled SARS-CoV-2-targeted antibody targets Spike S1 of SARS-CoV-2.

4. The method of claim 1, wherein the radiolabeled SARS-CoV-2-targeted antibody comprises [¹²⁵I]I-CR3022.

5. The method of claim 1, wherein the radiolabeled SARS-CoV-2-targeted antibody comprises iodine-125.

6. The method of claim 3, wherein the SARS-CoV-2-targeted antibody comprises CR3022.

7. The method of claim 4, wherein the diluted saliva sample comprises a volume ratio of salvia to the saline solution of between 1:7 to 1:11.

8. The method of claim 6, wherein the diluted saliva sample comprises a volume ratio of salvia to the saline solution of about 1:9.

9. The method of claim 6, wherein the saline solution comprises 1% BSA-PBS.

10. The method of claim 7, wherein the portion of the diluted saliva sample comprises about 500 μl diluted saliva sample.

11. The method of claim 10, wherein separating at least a portion of the target bound antibody from the unbound antibody in the first solution comprises gravity separation.

12. The method of claim 10, wherein separating at least a portion of the target bound antibody from the unbound antibody in the first solution comprises centrifugal separation.

13. The method of claim 12, comprising detecting the radiation level in the separated target bound antibody solution with a gamma counter.

14. A method for detecting SARS-CoV-2 virus in a patient, the method comprising:
contacting a diluted saliva sample comprising a saliva sample from a patient diluted with a saline solution, with a radiolabeled SARS-CoV-2-targeted antibody comprising [¹²⁵I]I-CR3022 to form a first solution comprising target bound antibody and unbound antibody;
separating at least a portion of the target bound antibody from the unbound antibody in the first solution to form a separated target bound antibody sample; and
detecting with a gamma counter a radiation level in the separated target bound antibody sample indicating the presence of the target bound antibody in the separated target bound antibody sample.

15. The method of claim 14, wherein the contacting the diluted saliva sample with the radiolabeled SARS-CoV-2-targeted antibody comprises contacting the diluted saliva sample with the radiolabeled SARS-CoV-2-targeted antibody in a separation tube, and the separating at least a portion of the target bound antibody from the unbound antibody in the first solution comprises separating at least a portion of the target bound antibody from the unbound antibody in the first solution in the separation tube using a centrifuge.

16. The method of claim 15, wherein the separation tube comprises a prepacked, single-use column.

17. The method of claim 16, wherein the separation tube is pretreated with a solution comprising 5% BSA-PBS.

18. A collection kit for collecting a saliva sample for use in the method of claim 14, the collection kit comprising:
instructions for collecting a saliva sample from a human patient and sending the saliva sample for analysis;
a sample collector for obtaining the saliva sample, the sample collector comprising a separation tube pretreated with saline solution; and
packaging for sending the saliva sample in the sample collector to a separate location for the analysis.

19. A process comprising
contacting a diluted saliva sample with a radiolabeled SARS-CoV-2-targeted antibody comprising [¹²⁵I]I-CR3022 to form a first solution comprising target bound antibody and unbound antibody, wherein the diluted saliva sample comprises the saliva sample from claim 18 diluted with a saline solution;
separating at least a portion of the target bound antibody from the unbound antibody in the first solution to form a separated target bound antibody sample; and
detecting with a gamma counter a radiation level in the separated target bound antibody sample indicating the presence of the target bound antibody in the separated target bound antibody sample.

20. The process of claim 19, wherein
contacting the diluted saliva sample with the radiolabeled SARS-CoV-2-targeted antibody comprises contacting the diluted saliva sample with the radiolabeled SARS-CoV-2-targeted antibody in a separation tube;
the separation tube comprises a prepacked, single-use column; and
separating at least a portion of the target bound antibody from the unbound antibody in the first solution comprises separating at least a portion of the target bound antibody from the unbound antibody in the first solution in the separation tube using a centrifuge.
